(19) United States
(12) Patent Application Publication
(10) Pub. No.: US 2011/0266220 A1
(43) Pub. Date: Nov. 3, 2011

(54) SEPARATIONS WITH HIGHLY SELECTIVE FLUOROPOLYMER MEMBRANES

(75) Inventors: Daniel Campos, Atglen, PA (US); Jonathan Lazzeri, Wilmington, DE (US); Stuart M. Nemser, Wilmington, DE (US)

(73) Assignee: CMS TECHNOLOGIES HOLDINGS, INC., Newport, DE (US)

(21) Appl. No.: 13/143,081

(22) PCT Filed: Jan. 5, 2010
(86) PCT No.: PCT/US10/20097

§ 371 Date: Jun. 30, 2011

Related U.S. Application Data
(60) Provisional application No. 61/143,007, filed on Jan. 7, 2009.

(51) Int. Cl. B01D 61/00; C07C 29/76; C07C 41/34; C07C 45/78; C07C 319/14; C07C 315/06; C07C 231/24; C07C 51/42; C07C 67/48; C07D 307/08; C07C 7/144; C10G 33/04; C08G 63/00; B01D 71/58; B01D 71/52; B01D 71/34; B01D 71/36; B01D 71/68; B01D 61/36

(52) U.S. Cl. 210/640; 210/651; 568/700; 568/579; 568/303; 568/18; 568/27; 568/28; 564/123; 562/608; 560/248; 568/916; 549/429; 564/216; 568/410; 585/818; 208/188; 528/271; 528/365; 210/490

(57) ABSTRACT

A method of separating components of mixtures of chemical compounds uses a nonporous membrane of copolymer of a perfluorinated cyclic or cyclizable monomer, and a 4 carbon dicarboxyl-containing comonomer, such as maleic anhydride. Optionally, the membrane composition includes an acyclic fluorinated olefin termonomer. The membranes provide a remarkably high selectivity of water relative to organic solvents and inorganic acids compared to dipolymer membranes of perfluorinated comonomers.

### CROSS REFERENCE TO RELATED APPLICATIONS

This application claims benefit of U.S. Provisional Application No. 61/143,007 filed Jan. 7, 2009.

### FIELD OF THE INVENTION

The invention relates to the separation of certain mixtures using selectively permeable membranes of highly fluorinated terpolymer composition. More specifically, it relates to separations of water from aqueous mixtures with organic compounds such as alcohols, acids, esters, ethers, amides, and hydrocarbon oils, or inorganic acids by selective membrane permeation in which the membrane composition comprises a polymer of a perfluorinated cyclic or cyclizable monomer a 4-carbon acid/anhydride comonomer, and, optionally, an acyclic fluorinated olefinic monomer.

### BACKGROUND OF THE INVENTION

In various important industrial chemical operations there are needs to effect certain separations of the mixtures such as, methanol/water, ethanol/water, n-propanol/water and isopropanol/water. Traditional membranes of diverse compositions have been applied to these separations. U.S. published patent application US2008099400A1 of Nemser et al., discloses the separation of water from mixtures with ethanol, mixtures of compounds with which water has a low relative volatility, and hydrocarbon oils. One membrane composition of great interest for separations such as these has been a dipolymer of perfluoro-2,2-dimethyl-1,3-dioxole (“PDD”) and tetrafluoroethylene (“TFE”), sold under the trademark Teflon® AF (DuPont, Wilmington, Del.). Better membrane separation performance than that obtained from these PDD/TFE dipolymers is desirable.

New PDD/TFE/maleic anhydride copolymers have been introduced. It has been now discovered that such compositions demonstrate surprisingly high selectivity in important separations compared to the conventional PDD/TFE copolymer compositions.

### SUMMARY OF THE INVENTION

The present invention provides a method of dehydrating diverse aqueous mixtures of chemical compounds by membrane separation in which the membrane comprises a nonporous, selectively permeable layer of a copolymer comprising two comonomers, namely, a perfluorinated cyclic or cyclizable monomer, and a4-carbon acid/anhydride. In a preferred embodiment, the method utilizes a membrane which further comprises an acyclic perfluorinated olefinic compound. Preferably the perfluorinated cyclic or cyclizable monomer is perfluoro-2,2-dimethyl-1,3-dioxole (“PDD”) and the 4-carbon acid/anhydride is maleic anhydride, maleic acid, fumaric acid or a combination thereof. A preferred acyclic perfluorinated olefinic compound is tetrafluoroethylene (“TFE”). The method can be applied to removing water from mixtures with a variety of organic compounds which have up to 12 carbon atoms and from mixtures with inorganic acids.

Accordingly this invention provides a method of dehydrating an aqueous mixture of chemical compounds comprising the steps of (i) providing a membrane comprising a nonporous, selectively permeable layer of a copolymer comprising copolymerized perfluorinated cyclic or cyclizable monomer, and a 4-carbon acid/anhydride, (ii) contacting the membrane with a liquid or vapor feed mixture of water and at least one other chemical compound, (iii) applying a driving force across the membrane effective to cause preferential permeation of water through the membrane, and (iv) recovering from the membrane a retentate composition depleted in water relative to the feed mixture, and (v) removing the permeate in the vapor phase.

The invention also provides a selectively permeable membrane useful for effecting removal of water from acqueous mixtures, the novel membrane having a polymeric selectively permeable layer comprising two comonomers, namely, a perfluorinated cyclic or cyclizable monomer, and a 4-carbon acid/anhydride, and optionally a third comonomer of an acyclic perfluorinated olefinic compound.

### BRIEF DESCRIPTION OF THE DRAWINGS

FIG. 1 is a semi-logarithmic plot of the water-to-ethanol selectivity, α, versus permeability of water, in barrers, for selected highly fluorinated polymeric selectively permeable membrane compositions.

FIG. 2 is a semi-logarithmic plot of the water-to-ethanol selectivity, α, versus glass transition temperature, Tg, (° C.), for selected highly fluorinated polymeric selectively permeable membrane compositions.

FIG. 3 is a semi-logarithmic plot of the selectivity of helium, carbon dioxide and oxygen, respectively, to nitrogen versus glass transition temperature, Tg (° C.), for selected highly fluorinated polymeric selectively permeable membrane compositions.

FIG. 4 is a schematic flow diagram of an apparatus utilized to determine the selectivities of membrane compositions to mixtures of chemical compounds.

### DETAILED DESCRIPTION OF THE INVENTION

This invention involves separation of mixtures effected by selectively permeable membranes of which the active selectively permeable component is a nonporous, amorphous copolymer. This copolymer comprises copolymerized perfluorinated cyclic or cyclizable monomer, and a 4-carbon-containing comonomer. A cyclizable monomer is an acyclic diene compound which can undergo ring formation during the polymerization process in which the copolymer according to this invention is formed. Preferably the perfluorinated cyclic or cyclizable monomer is selected from among perfluoro-2,2-dimethyl-1,3-dioxole (“PDD”), perfluoro-2-methylene-4-methyl-1,3,dioxolane (“PMD”), 2,2,4-trifluoro-5-trifluoromethoxy-1,3-dioxole (“TTD”) and perfluoro(4-vinyloxyl-1-butene) “PVOB”.

The 4-carbon-containing comonomer is an organic compound having dicarboxylic acid or anhydride functionality (hereinafter occasionally referred to as “4-carbon acid/anhydride” or “4CAA”). Preference is given to introducing the anhydride or dicarboxylic acid functionality using maleic anhydride. Maleic or fumaric acid may alternatively be employed. The anhydride and diacid groups in the copolymer backbone may be wholly or partially interconverted by hydration and dehydration.

The selectively permeable polymeric layer of the membrane can further comprise a third comonomer which is an acyclic fluorinated olefin compound. The acyclic fluorinated olefin is preferably selected from the group consisting of tetrafluoroethylene, chlorotrifluoroethylene, vinyl fluoride, vinylidene fluoride and trifluoroethylene More preferably the third comonomer is TFE. A greatly preferred selectively permeable polymeric layer of the novel membrane is a terpolymer of copolymerized PDD, TFE and 4CAA comonomers.

The fluorinated monomers are the predominant components of the active membrane layer composition. The 4CAA comonomer usually is present in minor proportions, i.e., less than 5 mole %, of all monomers. A preferred composition is a terpolymer in which the acyclic fluorinated olefin is TFE and in which molar ratio of perfluorinated cyclic or cyclizable monomer/TFE is about 60-50/40-50 with 4CAA being present in a complemental amount to total 100%. Preferably the polymerized 4CAA is about 0.1-4 mole %, more preferably about 0.2-1 mole %, and most preferably about 1 mole % of the active membrane layer composition. Preference is given to selectively permeable layer terpolymer compositions of about 54 mole % PDD/45 mole % TFE/1 mole % maleic anhydride and of about 59 mole % PDD/40 mole % TFE/1 mole % maleic anhydride.

The manufacture of a 54.7 mole % PDD/44.5 mole % TFE/0.8 mole % maleic anhydride terpolymer is disclosed in Example 15 of U.S. Pat. No. 6,423,798. The '798 patent teaches that fluorinated copolymers having 4CAA moiety grafted thereon are known to have a different structure from TFE/perfluorinated cyclic or cyclizable monomer/4CAA terpolymers. The former include the 4CAA moiety grafted onto an existing polymer such that the 4CAA moiety is usually a side chain and not part of the main polymer chain. The latter is a different structure in which the 4CAA moiety incorporates in the main polymer chain. The present invention can utilize fluorinated polymers in which the 4CAA moiety is a graft of a polymerized TFE/perfluorinated cyclic or cyclizable monomer structure. However, preference is given to separations in which the membrane active layer is polymerized TFE/perfluorinated cyclic or cyclizable monomer/4CAA comonomer in which the 4CAA moiety is in the main polymer chain.

The active selectively permeable component of the membrane for use in this invention is present as a nonporous polymeric film. The film can be a monolithic self-supporting structure, however usually it constitutes a layer of a multilayer composite structure in which the nonporous, selectively permeable layer is supported by a porous substrate. The physical membrane structure can be any of the well known configurations, such as flat sheet, hollow fiber, tubular, spiral wound and vortex devices (also known as “rotating” devices). Other useful configurations include pleated sheet and tube ribbon form. Membrane tubes and tube ribbons are disclosed in U.S. Pat. No. 5,565,166. Any porous substrate material offering support effective to maintain integrity of the active layer is suitable provided that the substrate is not degraded by the components and does not impede the transmission of the volatile component through the nonporous membrane. Representative examples of porous substrate material are polymers selected from the group consisting of polyacrylonitrile (“PAN”), polyether ether ketone (“PEEK”), polyvinylidene fluoride (“PVDF”), polytetrafluoroethylene (“PTFE”) and polysulfone (“PSF”).

Preferably the membrane structure takes the form of a hollow fiber membrane having a porous hollow fiber substrate material which bears a thin coating of the active layer on the inner, and/or outer surfaces of the fiber substrate. Typically, a plurality of hollow fiber membranes are bundled as a unit together within a single case such that the feed, permeate and retentate for all fibers in the unit flow through common feed, permeate and retentate stream ports, respectively. Such units are sometimes referred to as “modules”. Hollow fiber membranes and modules comprising hollow fiber membranes are well known as disclosed by U.S. Pat. Nos. 3,499,062 and 3,536,611, for example.

The novel selectively permeable membrane composition has applicability for separating components of diverse chemical mixtures. It is particularly useful for removing water from mixtures with many kinds of compositions such as mixtures of water with organic compounds, inorganic acids and combinations thereof. The organic compounds include typically small molecule hydrocarbon based-compounds and hydrocarbon oils and petroleum distillates. Small molecule hydrocarbon-based compounds include many liquid and vapor solvents and chemical reactant materials. Preferably these small molecule compounds have 2-12 carbon atoms. Types of organic compounds to which this invention is applicable include hydrocarbons, alcohols, acids, esters, ethers, ketones, sulfides, sulfoxides, sulfones and amides. Representative examples of organic compounds are acetic acid, ethyl acetate, ethanol, n-propanol, isopropanol, butanol, tetrahydrofuran, dimethyl formamide, dimethyl acetamide, dimethylsulfoxide, methyl ethyl ketone, methyl isobutyl ketone and petroleum distillate. Hydrocarbon oils include intermediate molecular weight (i.e., about 100-1000) hydrocarbon oligomers. Such compounds are typically formed by oligomerization of alpha olefin monomers having the structure C=CR_{f} in which R_{f} represents an aliphatic carbon radical having about 3-10 carbons atoms. Such hydrocarbon oils are often utilized in hydraulic power transmission fluid applications. The term “petroleum distillate” as used herein is meant to embrace individual hydrocarbon compounds or mixtures of hydrocarbons refined from crude oil, such as gasoline and other volatile fuels. Usually these mixtures include multiple components including various saturated and unsaturated compounds of linear-, cyclic-, and branched-carbon atom chains and aromatic compounds, and may include compounds having in excess of 12 carbon atoms. Representative examples of inorganic acids are nitric acid, sulfuric acid, and phosphoric acid.

The components other than water which constitute the mixtures subject to dehydration according to this invention can be liquid or vapor at ambient atmospheric conditions, i.e., about 27° C. and 1 atm pressure. The mixtures can be solutions, dispersions or both. The novel separation method by which water is removed from the mixtures is preferably vapor permeation. That is, the feed, retentate and permeate mixtures in contact with the membrane are in the vapor state and the components transferring through the membrane migrate by vapor permeation mechanisms. If any components of the mixture subject to separation by vapor permeation are in the liquid state, the feed stream to the membrane is vaporized prior to contacting the membrane. Usually the feed stream is heated to a temperature above the boiling point of the feed stream components at the feed stream pressure to vaporize the feed mixture.

The novel membrane according to this invention can also be utilized in membrane separation by the pervaporation method. In that technique, the feed mixture contacts the membrane in the liquid state, the migrating components transfer through the membrane and pass into the permeate which is in the vapor state.

### EXAMPLES

Polymers having compositions and physical properties presented in Table 1 were used in the following examples.

TABLE 1

| | | | | | | | Aver- |
|---|---|---|---|---|---|---|---|
| | | | | | Maleic | Film | age |
| | | | | | Anhy- | Den- | Thick- |
| Poly- | Tg | TFE | PDD | TTD | dride | sity | ness |
| mer | ° C. | mol % | mol % | mol % | mol % | g/cm³ | μm |
| AF2400¹ | 240 | 17 | 83 | 0 | 0 | 1.27 | 2.7 |
| AF1600¹ | 160 | 36 | 64 | 0 | 0 | 1.66 | 1.5 |
| AF1300¹ | 136 | 44 | 56 | 0 | 0 | 1.71 | 2.0 |
| A | 117 | 45³ | 55³ | 0 | 0.2-1³ | 1.68 | 2.8 |
| B | 126 | 45 | 55 | 0 | 0.2 | 1.72 | 2.0 |
| AD60X² | 104 | 40 | 0 | 60 | 0 | 1.83 | 2.2 |

¹Trademark Teflon ® (E. I. du Pont de Nemours & Co., Wilmington, Delaware)
²Trademark Hyflon ® (Solvay Fluorati Holding S.P.A. Italy)
³ Precise composition not known, however, best available information suggests composition to be about 45 mol % TFE, about 55 mol % PDD and about 0.2-1 mol % maleic anhydride

Film density was measured by the following procedure. A 0.5 wt % solution of each polymer dissolved in highly fluorinated Fluorinert® FC-770 Electronic Liquid solvent (3M Company), hereinafter “FC-770”, was poured into shallow glass pan and the solvent was allowed to dry at ambient temperature over about a 12 hour period. The air-dried films were removed from the pans and further dried in an oven at 120° C. for a similar length of time. From the films, 47 mm diameter disks were punched, weighed and measured for average thickness. Disk volumes were calculated based upon measured average thicknesses (approximately 20 μm) to provide density as ratio of measured weight per unit of thus calculated volume.

Composite membranes of the polymers identified in Table 1 were prepared as follows. Polymers were dissolved in FC-770 to form 0.5 wt % solutions. The solutions were sprayed onto 40 inch×16 inch sheets of porous polyacrylonitrile type PAN350 (Sepro Membranes Inc., Oceanside, Calif.) and air dried. Mass of the coated polymer was calculated from the difference between the initial and final weights of the coating solution utilized and the known polymer concentration. Average active layer membrane thickness was calculated as the coated polymer mass divided by the density (Table 1) divided by membrane area. Average active layer membrane thicknesses are shown in Table 1.

Disks of 142 mm diameter were punched from the composite membrane sheets for permeation testing. For each permeation test a disk was placed into a Pall model 11872 permeation cell. A pure gas of He, CO₂, O₂ or N₂ was fed to the cell at room temperature at pressures of 10, 20 and 30 psig, to determine steady state permeation rates. From these measurements the average pure gas permeabilities were determined. From these average permeabilities, selectivities of the gases relative to N₂ were calculated. N₂ permeability and O₂/N₂, He₂/N₂, CO₂/N₂ selectivities for each of the polymers are presented in Table 2.

TABLE 2

| | N₂ | | | |
|---|---|---|---|---|
| Poly- | Permeability | O₂/N₂ | He₂/N₂ | CO₂/N₂ |
| mer | (Barrer) | Selectivity | Selectivity | Selectivity |
| AF2400 | 461 | 2.1 | 5.7 | 5.2 |
| AF1600 | 149 | 2.6 | 14.4 | 5.8 |
| AF1300 | 55 | 2.9 | 23.8 | 7.1 |
| A | 57 | 2.8 | 21.1 | 6.5 |
| B | 64 | 2.9 | 20.7 | 6.8 |
| AD60X | 22 | 2.4 | 19.9 | 5.3 |

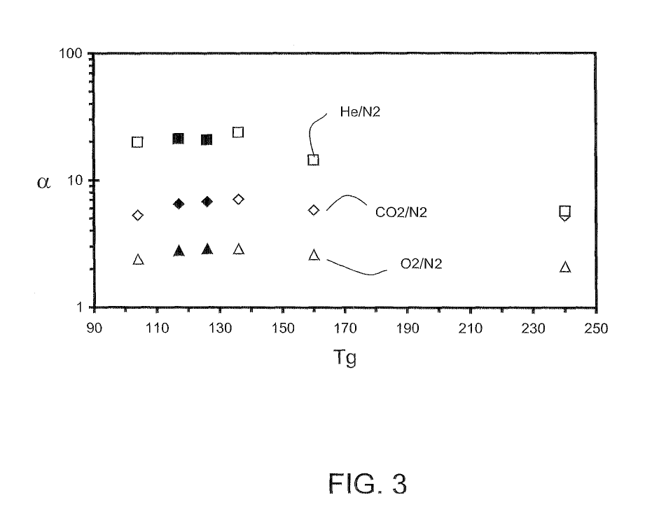

Gas pair selectivities, α, of Table 2 for each of the polymers are plotted against polymer Tg of Table 1 in a semi-logarithmic graph in FIG. 3. In this and the following plots, hollow symbols correspond to dipolymer membrane compositions and solid symbols correspond to terpolymer compositions A and B. All of the polymers generally provide a near-linear semi-logarithmic correlation having a trend of decreasing selectivity with increasing glass transition temperature for each of the gas pairs. For Tg of less than 140° C. the selectivities within each gas pair series are approximately the same. It is thus seen that the gas permeability characteristics of the 4ACC-containing terpolymer compositions A and B are comparable to those of similar perfluorinated dipolymer membranes with respect to these mixtures of gaseous components.

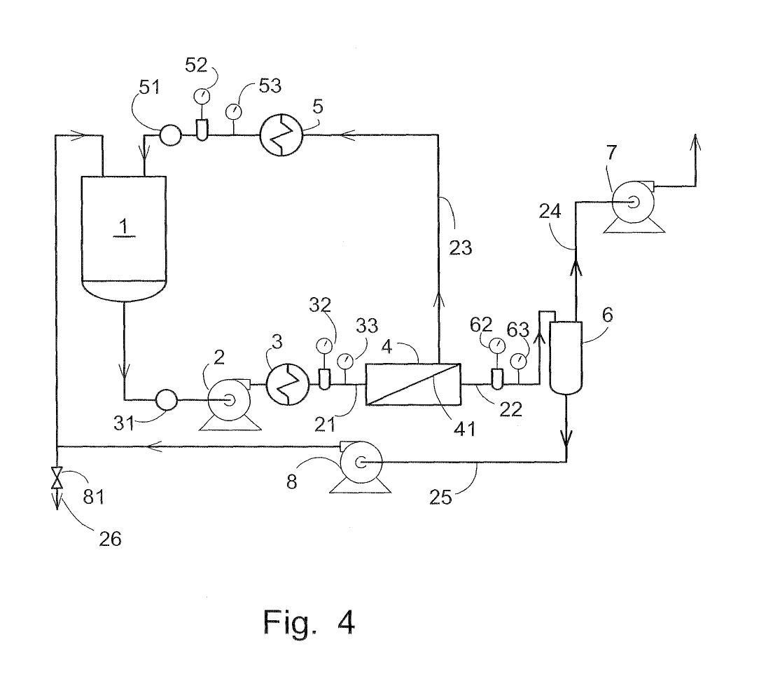

The same membrane samples used in the pure gas permeation tests were subjected to vapor permeation testing of an ethanol/water solution using the apparatus shown schematically in FIG. 4. An about 62 wt % ethanol/38 wt. % water solution was placed in feed tank 1 and circulated by feed pump 2 through the feed-retentate side of the 142 mm diameter disk permeation cell 4 containing membrane 41. Feed solution was drawn from feed tank 1 in the liquid state and completely vaporized by boiler 3 in feed transfer line 21 before contacting the membrane. Retentate was returned to the feed tank via transfer line 23. Retentate vapor in contact with the membrane was condensed completely by cooler 5 before re-entering tank 1. Permeate vapor 22 passing through membrane 41 was condensed in condenser 6 then returned via pump 8 in transfer line 25 to feed tank 1. Noncondensables from the permeate were withdrawn via transfer line 24 by vacuum pump 7 and exhausted to atmosphere. Densities of the liquid feed solution and condensed retentate were measured with ELITE® coriolis type flow sensor part #CMF010M (Micro Motion, Inc., Boulder, Colo.) inline analyzers 31, and 51, respectively. Temperatures of feed, retentate and permeate streams were measured by instruments 32, 52 and 62, respectively. Similarly, feed, retentate and permeate pressures were measured by gauges 33, 53, and 63, respectively.

After starting circulation through the permeation cell, flow was adjusted to achieve about 35 psia feed pressure on gauge 33 and boiler 3 was adjusted to heat the feed in line 21 to about 120° C. Permeate vapor was controlled to a subatmospheric pressure of 1.6 psia. Water permeated the membrane faster than ethanol to provide a water-rich permeate composition. The apparatus was permitted to operate steadily with recirculation of retentate and permeate to feed tank 1 while continuously monitoring pressure, temperature and density instrument indications. When the instrumentation indicated that steady state had been achieved, all temperature, pressure, and density meter conditions were recorded and valve 81 in sample line 26 was opened to obtain a small sample of condensed permeate for permeate flow rate measurement (i.e., weight collected per unit time). The sample was also subjected to off-line analysis of composition as a check of material balance calculations. Density and temperature measurements of the feed, retentate and permeate were used to provide corresponding ethanol and water concentrations based on known physical property data for ethanol/water solutions. Partial pressures of ethanol and water on the feed and permeate sides of the membrane were calculated from the determined component concentrations and measured stream pressures. Permeance of each component was calculated as the component permeate flowrate divided by the product of membrane area and difference between component feed and permeate partial pressures. Component permeabilities were calculated by multiplying permeance by average membrane thickness. Membrane selectivity was calculated as the ratio of the component permeabilities.

Water and ethanol permeabilities and water/ethanol selectivity determined by the foregoing procedure are presented for six membrane compositions in Table 3. Permeabilities are in units of Barrers. One barrer is equal to 1×10⁻¹⁰ cm³ (STP)·cm/(cm²·s·cmHg).

TABLE 3

| | H₂O | Ethanol | |
|---|---|---|---|
| Poly- | Permeability | Permeability | H₂O/Ethanol |
| mer | (Barrer) | (Barrer) | Selectivity |
| AF2400 | 7980 | 1157 | 6.9 |
| AF1600 | 2542 | 121 | 21 |
| AF1300 | 1902 | 60 | 32 |
| A | 2348 | 14 | 168 |
| B | 2170 | 38 | 57 |
| AD60X | 899 | 22 | 41 |

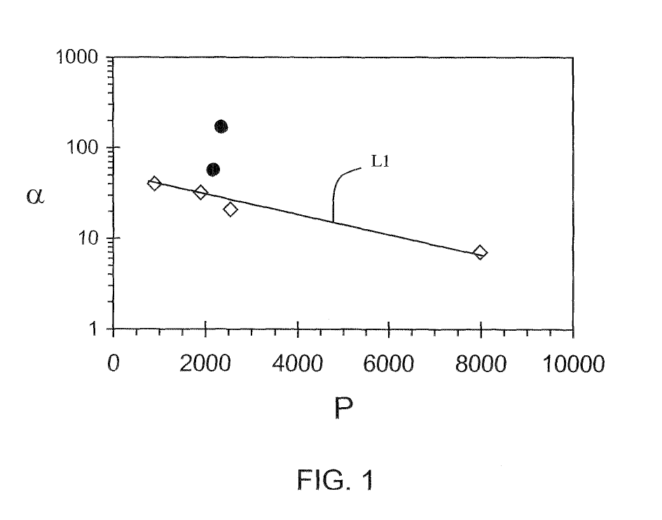

FIG. 1 is a graph of the water/ethanol selectivity, α, plotted on a logarithmic scale against water permeability in barrers from Table 3 data. The graph demonstrates that the dipolymer membrane compositions AF2400, AF1600, AF1300 and AD60X provide selectivities that lie predictably along a straight line L1. However, the membranes of terpolymers with a 4CAA comonomer have unusually higher water/ethanol selectivities than would be predicted. The high selectivity is very unexpected in view that the amount of TFE and perfluorinated cyclic or cyclizable monomer of membranes A and B are quite similar to AF1300 and AD60X membranes. The primary distinction of A and B membranes is that they incorporate less than 1 mole % of maleic anhydride comonomer. Although the amount of third comonomer is small, the membrane provides a remarkable increase in selectivity for water over ethanol. This seems to be caused by the very high water permeability for the A and B polymer membranes compared to the maleic anhydride-free dipolymer membranes. High water-to-ethanol selectivity makes the novel terpolymer membranes extremely useful for dehydrating aqueous mixtures.

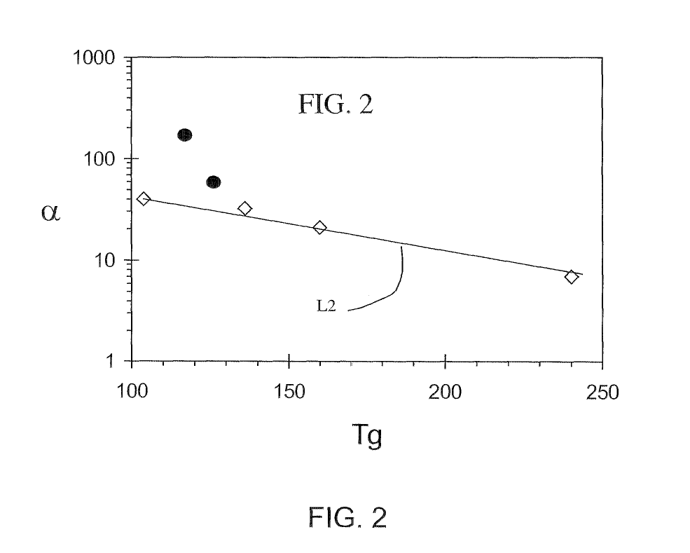

FIG. 2 is a graph of the same water/ethanol selectivity data from Table 3 plotted on a logarithmic scale against glass transition temperature, Tg, of the membrane polymer compositions from Table 1, FIG. 2 is thus comparable to FIG. 3. FIG. 2 shows that the selectivities of the maleic anhydride-free polymeric membranes lie along a semi-log straight line correlation, L2, similar to that seen in FIG. 3 for gas mixtures separated by the same membranes. However, the selectivities of the maleic anhydride-containing membrane polymers are unexpectedly higher and distant from line L2.

Although specific forms of the invention have been selected in the preceding disclosure for illustration in specific terms for the purpose of describing these forms of the invention fully and amply for one of average skill in the pertinent art, it should be understood that various substitutions and modifications which bring about substantially equivalent or superior results and/or performance are deemed to be within the scope and spirit of the following claims. The disclosures of every U.S. patent and U.S. published patent application identified herein is hereby incorporated by reference herein.

What is claimed is:

1. A method of dehydrating an aqueous mixture of chemical compounds comprising the steps of (i) providing a membrane comprising a nonporous, selectively permeable layer of a copolymer comprising copolymerized perfluorinated cyclic or cyclizable monomer, and a 4-carbon acid/anhydride, (ii) contacting the membrane with a feed mixture of water and at least one other chemical compound, (iii) applying a driving force across the membrane effective to cause preferential permeation of water through the membrane, and (iv) recovering from the membrane a retentate composition depleted in water relative to the feed mixture, and (v) removing the permeate in the vapor phase.

2. The method of claim 1 in which the copolymer further comprises an acyclic fluorinated olefinic monomer.

3. The method of claim 2 in which the acyclic fluorinated olefinic monomer is selected from the group consisting of tetrafluoroethylene, chlorotrifluoroethylene, vinyl fluoride, vinylidene fluoride and trifluoroethylene.

4. The method of claim 2 in which the 4-carbon acid/anhydride is selected from the group consisting of maleic anhydride, maleic acid, fumaric acid and a combination thereof.

5. The method of claim 4 in which the perfluorinated cyclic or cyclizable monomer is selected from the group consisting of perfluoro-2,2-dimethyl-1,3-dioxole (“PDD”), perfluoro-2-methylene-4-methyl-1,3,dioxolane (“PMD”), 2,2,4-trifluoro-5-trifluoromethoxy-1,3-dioxole (“TTD”) and perfluoro(4-vinyloxyl-1-butene) “PVOB”.

6. The method of claim 5 in which the perfluorinated cyclic or cyclizable monomer is perfluoro-2,2-dimethyl-1,3-dioxole (“PDD”).

7. The method of claim 6 in which the acyclic fluorinated olefinic monomer is tetrafluoroethylene.

8. The method of claim 5 in which the perfluorinated cyclic or cyclizable monomer is perfluoro-2-methylene-4-methyl-1,3,dioxolane (“PMD”).

9. The method of claim 5 in which the perfluorinated cyclic or cyclizable monomer is 2,2,4-trifluoro-5-trifluoromethoxy-1,3-dioxole (“TTD”).

10. The method of claim 5 in which the perfluorinated cyclic or cyclizable monomer is perfluoro(4-vinyloxyl-1-butene) “PVOB”.

11. The method of claim 2 in which the chemical compound is an organic compound selected from the group consisting of hydrocarbon oils, petroleum distillates, hydrocarbons, alcohols, acids, esters, ethers, ketones, sulfides, sulfoxides, sulfones and amides.

12. The method of claim 11 in which the organic compound has 2-12 carbon atoms.

13. The method of claim 11 in in which the organic compound is selected from the group consisting of acetic acid, ethyl acetate, ethanol, n-propanol, isopropanol, butanol, tetrahydrofuran, dimethyl formamide, dimethyl acetamide, dimethylsulfoxide, methyl ethyl ketone, methyl isobutyl ketone, hydrocarbon oils and petroleum distillate.

14. The method of claim 2 in which the chemical compound is an inorganic acid.

15. The method of claim 14 in which the inorganic acid is selected from the group consisting of nitric acid, sulfuric acid, and phosphoric acid.

16. The method of claim 2 in which the copolymer comprises from about 50 to about 83 mole percent of the perfluorinated cyclic or cyclizable monomer, from about 0.1 to 4 mole percent of the 4-carbon acid/anhydride, and a complemental amount to total 100 mole percent of tetrafluoroethylene.

17. A selectively permeable membrane comprising a nonporous layer of copolymer consisting essentially of (a) copolymerized perfluorinated cyclic or cyclizable monomer and (b) a 4-carbon acid/anhydride.

18. (canceled)

19. (canceled)

20. The membrane of claim 17 in which the 4-carbon acid/anhydride is selected from the group consisting of maleic anhydride, maleic acid, fumaric acid and a combination thereof.

21. The membrane of claim 17 in which the perfluorinated cyclic or cyclizable monomer is selected from the group consisting of perfluoro-2,2-dimethyl-1,3-dioxole (“PDD”), perfluoro-2-methylene-4-methyl-1,3,dioxolane (“PMD”), 2,2,4-trifluoro-5-trifluoromethoxy-1,3-dioxole (“TTD”) and perfluoro(4-vinyloxyl-1-butene) “PVOB”.

22. The membrane of claim 21 in which the perfluorinated cyclic or cyclizable monomer is perfluoro-2,2-dimethyl-1,3-dioxole (“PDD”).

23. (canceled)

24. The membrane of claim 21 in which the perfluorinated cyclic or cyclizable monomer is perfluoro-2-methylene-4-methyl-1,3,dioxolane (“PMD”).

25. The membrane of claim 21 in which the perfluorinated cyclic or cyclizable monomer is 2,2,4-trifluoro-5-trifluoromethoxy- 1,3-dioxole (“TTD”).

26. The membrane of claim 21 in which the perfluorinated cyclic or cyclizable monomer is perfluoro(4-vinyloxyl-1-butene) “PVOB”.

27. (canceled)

28. (canceled)

29. (canceled)

30. (canceled)

31. (canceled)

32. (canceled)

33. The membrane of claim 17 in which the nonporous layer is positioned in direct contact on one side of a porous layer of a substrate selected from the group consisting of polyacrylonitrile (“PAN”), polyether ether ketone (“PEEK”), polyvinylidene fluoride (“PVDF”), polytetrafluoroethylene (“PTFE”) and polysulfone (“PSF”).

34. The method of claim 1 in which the feed mixture contacting the membrane is in the vapor state.

35. The membrane of claim 20 in which the perfluorinated cyclic or cyclizable monomer is perfluoro-2,2-dimethyl-1,3-dioxole (“PDD”).

36. A method of dehydrating an aqueous mixture of chemical compounds comprising the steps of (i) providing a membrane comprising a nonporous, selectively permeable layer of a copolymer comprising copolymerized perfluoro-2,2-dimethyl-1,3-dioxole, and a 4-carbon acid/anhydride, (ii) contacting the membrane with a feed mixture of water and at least one other chemical compound, (iii) applying a driving force across the membrane effective to cause preferential permeation of water through the membrane, and (iv) recovering from the membrane a retentate composition depleted in water relative to the feed mixture, and (v) removing the permeate in the vapor phase.

37. The method of claim 36 in which the 4-carbon acid/anhydride is selected from the group consisting of maleic anhydride, maleic acid, fumaric acid and a combination thereof.

38. The method of claim 37 in which the chemical compound is an organic compound selected from the group consisting of hydrocarbon oils, petroleum distillates, hydrocarbons, alcohols, acids, esters, ethers, ketones, sulfides, sulfoxides, sulfones and amides.
